(19) United States
(12) Patent Application Publication
(10) Pub. No.: US 2025/0383238 A1
(43) Pub. Date: Dec. 18, 2025

(54) ACTIVATABLE TEMPERATURE EXPOSURE INDICATOR AND METHOD OF MANUFACTURING SAME

(71) Applicant: Temptime Corporation, Morris Plains, NJ (US)

(72) Inventors: Eric W. LIBERATO, Pequannock, NJ (US); John Olson, Dayton, NJ (US); Mohannad ABDO, Clifton, NJ (US); Brian S. HUFFMAN, Belle Mead, NJ (US); Robert REILLY, Columbia, NJ (US)

(73) Assignee: Temptime Corporation, Morris Plains, NJ (US)

(21) Appl. No.: 18/747,056

(22) Filed: Jun. 18, 2024

(51) Int. Cl. G01K 1/02; G01K 3/04; G01K 11/125

(52) U.S. Cl. CPC G01K 1/02; G01K 3/04; G01K 11/125

(57) ABSTRACT

Formulations to support activatable visual indicator platforms is disclosed herein. An example formulation to support activatable visual indicator platforms includes an activatable temperature exposure indicator having a response temperature, including a substrate, a wick physically coupled to or contained in the substrate, an indicator region, on or adjacent to a first end of the wick, and a bonding material containing a plurality of microcapsules disposed on or adjacent to a second end of the wick, opposite the first end.

### BACKGROUND

Many vaccines, drugs, foodstuffs, and other products are temperature-sensitive, or perishable, and can lose quality with time at rates that are influenced by ambient temperatures. Time-temperature indicators are known which can provide a simple visual indication of the cumulative historical exposure of a host product to heat or exposure to a peak temperature for even a short period of time. The visual indication can be used to provide a signal of when a product may have lost quality or freshness.

Known time-temperature indicators can provide a color change at a predetermined threshold or end point to indicate possible loss of quality or freshness of the host product. The color change can be displayed in a suitable label, or the like, to be read optically, for example, visually by a human viewer. The color change can be chromatic or achromatic or provided by another visually detectable optical parameter change. The temperature-response parameters of the time-temperature indicator can be correlated with a deterioration characteristic of the host product to coordinate the color change appropriately with the likely condition of the host product.

Also, certain perishable products, for example, vaccines and sensitive medications as well as some foodstuffs and other products including some industrial products can have their quality or safety compromised by relatively brief exposures to a temperature in excess of a predetermined threshold. Some indicators are activatable, that is the indicator does not operate to detect exposure until after an activation event occurs. This may help avoid the need to store such indicators in chilled condition prior to pairing them with a product to be monitored. Said differently, if a thermal indicator is to be installed on a host product, the indicator must be held below the temperature which the thermal indicator is configured to indicate prior to installation on the host product. If a sufficient thermal exposure were to occur, the indicator would transition to an indicative state prior to installation, and provided the indicator is a single-use indicator, the indicator would be expended prior to intended use. For example, indicators configured for use with refrigerated items, e.g., indicators showing when host products have warmed above a refrigerator temperature, generally need to be refrigerated prior to being paired with a host product, which results in an additional cost to the user and complications in inventory management and manufacturing processes.

### SUMMARY

In a first embodiment, the technology of the present disclosure is provided by an activatable temperature exposure indicator having a response temperature, including a substrate, a wick physically coupled to or contained in the substrate, an indicator region, on or adjacent to a first end of the wick, and a bonding material containing a plurality of microcapsules disposed on or adjacent to a second end of the wick, opposite the first end. The bonding material has a liquefaction temperature higher than the response temperature. Each microcapsule of the plurality of microcapsules contains an indicator material microencapsulated in an activatable shell. The indicator material is configured to liquefy responsive to exposure to a temperature at or above the response temperature. The activatable shells are configured to contain the indicator material when liquefied and are configured to rupture in response to an application of an activation action exceeding a predetermined activation threshold, releasing the indicator material. The indicator material is configured to permeate pores of the wick and migrate along the wick into the indicator region when liquefied and released from the microcapsules, producing an observable change in the indicator region. The bonding material couples the microcapsules to the wick without filling pores of the wick and the bonding material facilitates migration of liquefied indicator material into the wick, and blocks migration of the microcapsules into the wick.

In a variation of this embodiment, the wick and the bonding material containing the microcapsules are laminated between the substrate and a sealing layer.

In a variation of this embodiment, the bonding material containing the microcapsules is disposed in a reservoir contacting the second end of the wick.

In a variation of this embodiment, the bonding material containing the microcapsules is disposed atop the second end of the wick.

In a variation of this embodiment, the indicator region comprises a portion of the first end of the wick.

In a variation of this embodiment, the indicator region comprises a reservoir contacting the first end of the wick.

In a variation of this embodiment, the observable change is a change in a color state selected from a group consisting of a change of reflectivity, a change in transparency, a change in hue, a change in chroma, a change in apparent color, and combinations thereof.

In a variation of this embodiment, the observable change is a change in an electrical property selected from a group consisting of a change in conductivity, a change in resistance, a change in impedance, a change in capacitance, and combinations thereof.

In a variation of this embodiment, the activation action is a compressive stress, and the predetermined activation threshold is selected from a group consisting of a stress exceeding 0.1 psi a stress exceeding 0.5 psi, a stress exceeding 1 psi, a stress exceeding 2 psi, a stress exceeding 5 psi, a stress exceeding 10 psi, and a stress exceeding 15 psi.

In a variation of this embodiment, the activation action is a shear stress, and the predetermined activation threshold is selected from a group consisting of a stress exceeding 0.1 psi a stress exceeding 0.5 psi, a stress exceeding 1 psi, a stress exceeding 2 psi, a stress exceeding 5 psi, a stress exceeding 10 psi, and a stress exceeding 15 psi.

In a variation of this embodiment, the activation action is a thermal stress combined with a compression stress or shear stress, the thermal stress configured to reduce the predetermined activation threshold of the compression stress or the shear stress.

In a variation of this embodiment, the bonding material comprises a material selected from a group consisting of a polymer having side-chain crystallinity, polymeric materials, an alkane, a wax, an alkane wax, esters, and combinations thereof.

In a variation of this embodiment, the indicator material is selected from a group consisting of a polymer having side-chain crystallinity, polymeric materials, an alkane, a wax, an alkane wax, dyes, leuco dyes, chemical pigments, particles containing copper, particles containing silver, particles containing graphite, particles containing conductive metals, particles containing conductive non-metal materials, and combinations thereof.

In a variation of this embodiment, the activatable shells comprise a material selected from a group consisting of a protein, a gel, a polyurea formaldehyde, a polymelamine formaldehyde, a wax material, an emulsion, and combinations thereof.

In a variation of this embodiment, the response temperature for the indicator material is defined within a range bounded by −5 degrees Celsius (C) and 35 degrees C.

In a variation of this embodiment, the liquefaction temperature of the bonding material is greater than the response temperature of the activatable temperature exposure indicator.

In a variation of this embodiment, the liquefaction temperature of the bonding material is defined within a range bounded by 40 degrees Celsius (C) and 115 degrees C.

In a variation of this embodiment, each microcapsule of the plurality of microcapsules has diameter length in a range of 20 micrometers (μm) to 750 μm.

In a second embodiment, the technology of the present disclosure is provided by a method of making an activatable temperature exposure indicator, including placing a wick on a substrate, and dispensing a bonding material proximately to a wick. A plurality of microcapsules is dispersed within the bonding material, each microcapsule including an indicator material contained within an activatable shell. The bonding material containing the microcapsules is dispensed at a dispensation temperature above a liquefaction temperature of the bonding material, such that the bonding material is dispensed in a liquid phase, and the bonding material containing the microcapsules transitions out of the liquid phase after being dispensed and contacting the wick, and the bonding material couples the microcapsules to the wick without filling pores of the wick, and the bonding material blocks migration of the microcapsules into the wick.

In a variation of this embodiment, the liquefaction temperature of the bonding material is greater than a response temperature of the indicator material and defined within a range bounded by 40 degrees C. and 120 degrees C.

In a variation of this embodiment, the method further includes laminating the wick and the bonding material containing the microcapsules between the substrate and a sealing layer.

In a variation of this embodiment, the bonding material comprises a material selected from a group consisting of a polymer having side-chain crystallinity, polymeric materials, an alkane, a wax, an alkane wax, esters, and combinations thereof.

In a variation of this embodiment, the indicator material is configured to liquefy responsive to an exposure to a temperature above a predetermined response temperature defined within a range bounded by −15 degrees Celsius (C) and 35 degrees C.

In a variation of this embodiment, the activatable shells are configured to contain the indicator material when liquefied.

In a variation of this embodiment, the activatable shells are configured to rupture in response to an application of an activation action exceeding a predetermined activation threshold, releasing the indicator material.

In a variation of this embodiment, the indicator material is configured to permeate pores of the wick and migrate along the wick into an indicator region when liquefied and released from the microcapsules, producing an observable change in the indicator region.

In a variation of this embodiment, the activation action is a compression stress with a predetermined activation threshold selected from a group consisting of a stress exceeding 0.1 psi a stress exceeding 0.5 psi, a stress exceeding 1 psi, a stress exceeding 2 psi, a stress exceeding 5 psi, a stress exceeding 10 psi, and a stress exceeding 15 psi.

In a variation of this embodiment, the activation action is a shear stress with a predetermined activation threshold selected from a group consisting of a stress exceeding 0.1 psi a stress exceeding 0.5 psi, a stress exceeding 1 psi, a stress exceeding 2 psi, a stress exceeding 5 psi, a stress exceeding 10 psi, and a stress exceeding 15 psi.

In a variation of this embodiment, the indicator material is selected from a group consisting of a polymer having side-chain crystallinity, polymeric materials, an alkane, a wax, an alkane wax, dyes, leuco dyes, chemical pigments, particles containing copper, particles containing silver, particles containing graphite, particles containing conductive metals, particles containing conductive non-metal materials, and combinations thereof.

In a variation of this embodiment, the activatable shells comprise a material selected from a group consisting of a protein, a gel, a polyurea formaldehyde, a polymelamine formaldehyde, a wax material, an emulsion, and combinations thereof.

In a variation of this embodiment, each microcapsule of the plurality of microcapsules has a diameter length in a range of 20 micrometers (μm) to 750 μm.

In a variation of this embodiments, the wick comprises a laminate layer and a wicking material, and the method further includes cutting a laminated sheet of the laminate layer and the wicking material into a plurality of wicks; picking the wick from the plurality of wicks using a pick and place device; and placing the wick of the plurality of wicks onto the substrate using the pick and place device.

In a third embodiment, the technology of the present disclosure is provided by A method of making a plurality of activatable temperature exposure indicators including laminating an article of wicking material with a structural layer, forming a laminated sheet, cutting the laminated sheet into a plurality of wicks, placing cach wick of the plurality of wicks onto a respective substrate, and dispensing a bonding material proximately to each wick. A plurality of microcapsules is dispersed within the bonding material, each microcapsule including an indicator material contained within an activatable shell. The bonding material containing the microcapsules is dispensed at a dispensation temperature above a liquefaction temperature of the bonding material, such that the bonding material is dispensed in a liquid phase. The bonding material containing the microcapsules transitions out of the liquid phase after being dispensed and contacting the wick. The bonding material couples the microcapsules to the wick without filling pores of the wick, and the bonding material blocks migration of the microcapsules into the wick.

In a variation of this embodiment, the method further includes laminating cach wick and the bonding material containing the microcapsules between the respective substrate and a scaling layer.

In a variation of this embodiment, the respective substrate is a region on an article of substrate material demarcated into a plurality of regions.

In a variation of this embodiment, the article of substrate material is a roll of substrate material.

In a variation of this embodiment, the method further includes unwinding the roll of substrate material, exposing a respective substrate prior to a wick being placed on the respective substrate, such that the activatable temperature exposure indicators made with the roll of substrate material are connected.

In a variation of this embodiment, the method further includes winding the connected activatable temperature exposure indicators into a roll of activatable temperature exposure indicators.

In a fourth embodiment, the technology of the present disclosure is provided by an activatable environmental exposure indicator having a response condition, including a substrate, a wick physically coupled to or contained in the substrate, an indicator region, on or adjacent to a first end of the wick, and a bonding material containing a plurality of microcapsules disposed on or adjacent to a second end of the wick, opposite the first end. Each microcapsule of the plurality of microcapsules contains an indicator material microencapsulated in an activatable shell. The indicator material is configured to liquefy responsive to exposure to the response condition. The activatable shells are configured to contain the indicator material when the liquefied. The activatable shells are configured to rupture in response to an application of an activation action exceeding a predetermined activation threshold, releasing the indicator material. The indicator material is configured to permeate pores of the wick and migrate along the wick into the indicator region when liquefied and released from the microcapsules, producing an observable change in the indicator region. The bonding material couples the microcapsules to the wick without filling pores of the wick, and the bonding material facilitates migration of liquefied indicator material into the wick, and blocks migration of the microcapsules into the wick.

In a variation of this embodiment the response condition is selected from a group consisting of a temperature excursion above a predetermined temperature, a temperature excursion above a predetermined temperature threshold for at least a predetermined amount of time, a temperature excursion below a predetermined temperature, a temperature excursion below a predetermined temperature for at least a predetermined amount of time, cumulative exposure to temperature over a time period above a predetermined threshold for at least a predetermined amount of time, an exposure to a particular chemical, an oxygen exposure, an ammonia exposure, an exposure to a particular chemical above a threshold concentration, an exposure to a particular chemical above the threshold concentration for at least a predetermined amount of time, an exposure to at least a predetermined amount of radiation of a particular type, an predetermined electromagnetic exposure, a humidity exposure, an exposure to a humidity level above a predetermined threshold, and an exposure to a humidity level above a predetermined threshold for at least a predetermined amount of time.

### BRIEF DESCRIPTION OF THE DRAWINGS

The accompanying figures, where like reference numerals refer to identical or functionally similar elements throughout the separate views, together with the detailed description below, are incorporated in and form part of the specification, and serve to further illustrate embodiments of concepts that include the claimed invention, and explain various principles and advantages of those embodiments.

FIG. 1 illustrates a microcapsule, according to embodiments of the present disclosure.

FIG. 2 illustrates a bonding material in which the microcapsules of FIG. 1 are disposed, according to embodiments of the present disclosure.

FIG. 3 illustrates an activatable indicator in an unactivated state, according to embodiments of the present disclosure.

FIG. 4 illustrates the activatable indicator of FIG. 3 in an activated state, according to embodiments of the present disclosure.

FIG. 5 illustrates the activatable indicator of FIG. 3 in an activated and exposed state, according to embodiments of the present disclosure.

FIG. 6 illustrates the activatable indicator of FIG. 3 in an activated and exposed state, where an indicator material is in an indicator region of the activatable indicator, according to embodiments of the present disclosure.

FIG. 7 illustrates another activatable indicator according to embodiments of the present disclosure.

FIG. 8 illustrates a flowchart for a method of producing an indicator, according to embodiments of the present disclosure.

FIG. 9 illustrates a flowchart of a method for producing a plurality of indicators, according to embodiments of the present disclosure.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

The apparatus and method components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

### DETAILED DESCRIPTION

Notwithstanding the foregoing proposals for cumulative and threshold temperature indicators, it would be desirable to have a relatively simple activatable threshold or time temperature indicator having enhanced response characteristics, such as a delayed response, a simple method of manufacture, and a minimal space requirement.

To overcome one or more of the drawbacks of known environmental exposure indicators, the present disclosure discusses activatable environmental exposure indicators intended to be associated with a host product to monitor the exposure of the host product to ambient temperatures.

The environmental exposure indicators discussed herein generally utilize activatable microcapsules containing a liquefiable indicator material, such that the indicator material liquefies in response to a temperature at and/or above the melting point of the indicator material. Prior to activation, the microcapsules contain the indicator material, even when liquified. Once the microcapsules are activated, the indicator material, when liquified, is able to migrate into and along a wick.

The environmental exposure indicators discussed herein generally utilize a bonding material in which the activatable microcapsules may be dispersed, disposed proximate, in contact with, near, and/or on top of the wick. The bonding material maintains a proximity of the indicator material to the wick, such that when the indicator material is released from the microcapsule and liquefied, the indicator material absorbs, diffuses, wicks, or otherwise migrates into the wick, substantially uninhibited by the bonding material. The bonding material is configured and applied such that the microcapsules are held proximately to the wick, but the bonding material is not drawn into the wick in a manner which would preclude the wicking of the indicator material. In some cases, the bonding material is configured to have a viscosity that is too great for the bonding material to be absorbed by the wick. In some cases, and as is described by methods discussed herein, the bonding material is dispensed in a liquid state at a temperature marginally in excess of the solidification temperature of the bonding material, such that the bonding material rapidly solidifies upon contacting the wick, prior to potentially permeating the wick.

When the microcapsules are activated, e.g., by rupturing or breaking the microcapsules, the liquefied indicator material is able to migrate into the wick, permeating pores of the wick, and being transported along the wick to an indicator region, thus producing an observable effect in the indicator region.

The term “observable effect” can pertain to a change in color state or other appearance of the indicator region, or a change in electrical state of the indicator region. The term “change in color state” may refer a change in color, hue, chroma, or apparent color, a change from a darker to a lighter appearance, a change from a lighter to a darker appearance, a change in opacity (e.g. a change in transparency), or a change in brilliance. The term “color” is used herein to include achromatic visual appearances such as black, gray, and white, as well as primary, secondary, and other color hues, such as, without limitation, red, yellow, green, blue, purple, orange, brown, and other hues. The term “change in electrical state” may refer to a change in conductivity, resistance, impedance, resistivity, or capacitance, as may be measured across the indicator region.

One potential advantage of the technology of the present disclosure is to develop an indicator that will respond to an ascending environmental exposure temperature excursion only after the indicator (and by implication a host product) has been exposed to a temperature above an environmental exposure temperature threshold for a specified time period.

The environmental exposure indicators discussed herein and contemplated by the authors of this disclosure utilize a liquefiable material that can be configured to react to an environmental exposure temperature above a predetermined threshold relatively quickly. This is because the liquefiable material of some embodiments is configured or selected to have a sharp melting point, such that liquefaction happens very quickly over a small temperature range. Thus, exposure to an environmental exposure or peak temperature exceeding the melting point of the liquefiable material causes a quick state change. However, notwithstanding a relatively quick response by the liquefiable material to heat, some indicators discussed herein exhibit a time-dependent response that halts below the environmental exposure temperature threshold and resumes again in an additive manner. Again, in some embodiments, this is due to the liquefiable material having a sharp transition between a liquid phase and a solid phase.

In other words, where an indicator is configured to signal a response after an exposure of about 30 minutes at and/or above the environmental exposure temperature threshold, a 20-minute exposure will not trigger a response in the indicator region, but if the indicator is again exposed to a temperature at and/or above the environmental exposure temperature threshold, only about ten more minutes of exposure will yield a visual response in the indicator region. In some embodiments as noted above, this behavior is achieved because the liquefiable solid (such as a side-chain crystalline polymer) readily crystallizes and within a narrow temperature range. Once the environmental exposure temperature has been exceeded, a drop in temperature below the environmental exposure will cause almost immediate cessation of the time-dependent response. The response will resume once the environmental exposure temperature threshold is again exceeded.

As used herein, the terms “environmental exposure” and “environmental exposure temperature threshold” have an understood meaning in the art and include a temperature, usually a temperature above 0° C. (though temperatures below 0° C. are also contemplated), that can cause damage or harm to a product, such as a food or a vaccine that may require refrigeration to avoid spoilage or maintain efficacy for extended periods. The term “environmental exposure temperature threshold,” then, can include any predetermined temperature that is above a desired storage temperature of a perishable product, though in some cases exposure for short periods of time may not damage or harm a particular product. Thus, some embodiments disclosed herein are configured to provide signal of exposure to temperatures at and/or above an environmental exposure temperature threshold only after a specified amount of time even if exposure occurs at different times.

In some embodiments, the liquifiable material has a “sharp” liquefaction point, meaning that the transition from solid to liquid happens very quickly over a very small temperature range. In some embodiments, liquefaction temperature and solidification temperature of the liquifiable solid are identical. In some embodiments, the liquefaction and solidification temperatures are within about 0.1° C., within about 0.5° C., within about 1.0° C., within about 1.5° C., within about 2° C., within about 2.5° C., within about 3.0° C., within about 3.5° C., within about 4.0° C., within about 4.5° C., within about 5° C., or within about 10° C. of each other.

As used herein, the term “solid phase” may refer to a material in a non-liquid state such that the material is incapable of fluid flow. In some examples “solid phase” may refer to a gelled state, a highly viscous state, a truc solid state, and the like. Similarly, the terms “solidification” and “solidify” are used to describe the transition in which a material not in the solid phase enters the solid phase. The terms “solidification point” and “solidification temperature” are used to describe a temperature, or temperature range, at or in which a material may undergo solidification.

As used herein, the term “liquid phase” is used to describe a state of a material in which the material is capable of fluid flow. Similarly, the terms “liquefaction” and “liquefy” are used to describe the transition in which a material not in the liquid phase enters the liquid phase. The terms “liquefaction point” and “liquefaction temperature” are used to describe a temperature, or temperature range, at or in which a material may undergo liquefaction.

Suitable liquifiable materials include synthetic polymeric materials that are solid below the threshold temperature and are, or can become, an amorphous solid or a viscous liquid when at and/or above a threshold temperature. Such synthetic polymeric materials are liquifiable. Useful synthetic polymers can also be hydrophobic, if desired. Suitable liquifiable materials include side-chain crystallizable polymers (e.g., poly(hexadecylmethacrylate)).

As used herein, the term “polymer”, and its linguistic variations, refers to copolymers, and higher order polymers, as well as homopolymers, unless the context indicates otherwise, for example, by describing or referencing one or more specific homopolymers.

When solid, the synthetic polymeric material can be crystalline or partially crystalline. Crystalline or partially crystalline synthetic polymeric materials can have desirably sharp transitions from a solid state to a liquid state.

Side chain (liquid) crystalline polymers (abbreviated as SCC hereafter) are particularly suitable liquifiable materials, though other suitable materials such as waxes could readily be used. SCC polymers have a conventional polymer backbone and side chains that can co crystallize. Typically, they are chains that have six or more carbons with a crystallization temperature that is, therefore, adjustable. In some embodiments, the side chains “melt” independently of the main polymer chain so that the phenomenon can be used to release other materials that have been encapsulated within the overall polymer structure. Another advantage of SCC polymers is that their molecular weight and degree of crosslinking can be adjusted to control their physical properties including their permeability and in turn provide an approach to tailor the time delay.

Some embodiments discussed here utilize two or more compounds capable of reacting with each other to yield a color change. The two or more reactants may separate, and contained in distinct microcapsules, which substantially prevent the compounds from interacting prior to the activation of the microcapsules. Alternatively, a first of the compounds may be contained in the microcapsules, and a second of the compounds may be disposed in the indicator region of the indicator. An indicator can be assembled in such a manner as to prevent contact between the reactive compounds during the manufacturing process. When the indicator is heated above the liquefaction temperature of the liquefiable material and the microcapsules are activated, migration of the reactive compound(s) into the wick can begin. However, in some embodiments, there is a time delay before color change is observed. The indicator will show a response but only after a specific time delay that can be controlled by adjusting a number of parameters such as, among other features, choice of liquefiable solid, thickness of the liquefiable solid, molecular weight of the liquefiable solid, presence or absence of a barrier layer, barrier layer thickness, physical aspects of the barrier layer, choice of barrier material, reactant concentration, presence of catalysts, and choice of reactants.

Used in combination with the liquifiable material, in some embodiments, are color-reacting materials, such as two reactants kept separate by the microcapsules but allowed to react with each other after activation or migration. Dyes can also be dissolved in such liquifiable materials to provide an intense color. In some embodiments, the color-reacting materials, or color-forming reactants, produce a distinct color change or change in opacity when brought into contact with each other.

When the reactants come into contact, the appearance change of the indicator may be to go from clear to black, from clear to a dark color, from a light color to a dark color, from a light color to black, etc. In some embodiments, a background is visible through the liquifiable layer(s) prior to the reaction, thereby indicating that the predetermined temperature threshold and required exposure period have not yet been satisfied. The background may include words, numbers, or a pattern, or may simply comprise a color that is easily obscured by the color-forming reaction of the reactants. In some embodiments, a pattern on the background is at least partially obscured by the light color of the liquifiable layer(s), and the pattern becomes more visible after the color-forming reaction. For example, if the pattern is formed with an ink having a color similar to the color of the pre-reacted reactants, a color change produced by the interaction of the color-forming reactants may render the pattern more visible.

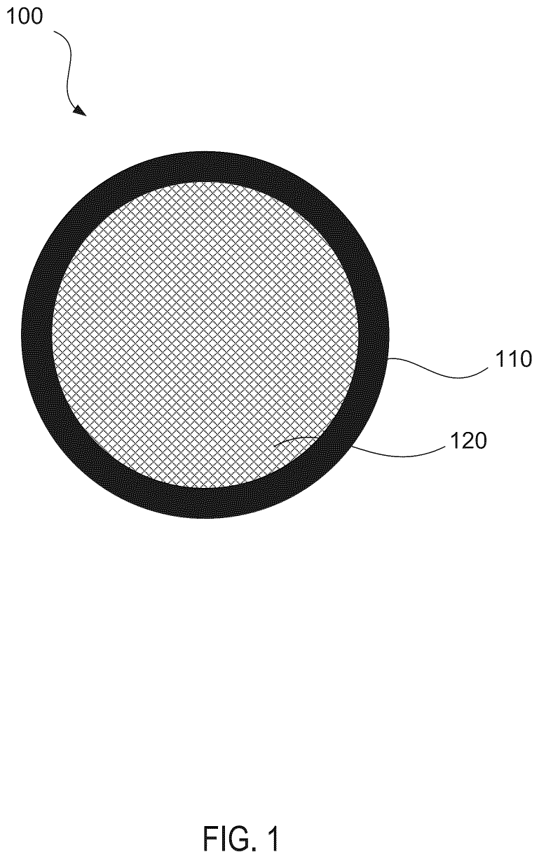

FIG. 1 illustrates a microcapsule 100 containing an indicator material 120 in an activatable shell 110, according to embodiments of the present disclosure. According to some embodiments, the indicator material 120 is a material configured to liquefy responsive to a predetermined environmental exposure and produce an observable effect either alone or in conjunction with other components and materials.

The indicator material 120 may be formed by a carrier material which is combined with an indicative material. The carrier material is a liquifiable material, which may be configured to liquefy (e.g., transition from a solid phase to a liquid phase) responsive to a predetermined environmental exposure, such that the indicator material 120 liquefies responsive to the predetermined environmental exposure. When the carrier material is liquefied, the indicator material 120 is capable of fluid flow, such that the indicative material is transported in the flow by the carrier material. According to some embodiments, the indicative material is a material that may be employed, either alone or in conjunction with other components and materials, to be combined with the carrier material and produce an observable effect when the carrier material is liquefied, indicating that the indicator material 120 has liquefied. According to some embodiments, the physical state of the indicative material does not effect the liquefaction or non-liquefaction of the carrier material. Thus, when the carrier material is in the liquid phase, the indicator material 120 is in the liquid phase, and when the carrier material is in the solid phase, the indicator material 120 is in the solid phase, and the indicator material 120 liquefies responsive to the predetermined environmental exposure. In some examples, a carrier material may possess indicative properties such that an additional indicative material is not necessitated, and the indicator material 120 includes only a carrier material.

The carrier material may be any such material capable of exhibiting a phase change (e.g. liquefying) from a substantially solid phase (e.g., solid, highly viscous, gelled) to a liquid phase upon the occurrence of a predetermined environmental stimulus (e.g., predetermined environmental exposure, response condition). As used herein, the term “solid phase” is used to describe the non-liquefied state of the carrier material, and may refer to a gelled state, a highly viscous state, or a solid state where the carrier material is incapable of fluid flow. As used herein, the term “liquid phase” is used to describe the liquefied state of the carrier material and refers to a state in which the transport material is capable of fluid flow. In some embodiments, there may be combinations of different indicator materials contained within microcapsules 100. This may include multiple types of microcapsules 100 containing multiple types of carrier materials. The different indicator materials may be placed into microcapsules 100 to keep the indicator materials 120 from premature contact with each other.

The carrier material, and thus the indicator material, may be configured to liquefy responsive to an exposure to a temperature above a response temperature threshold defined within a range bounded by −15 degrees C. and 35 degrees C.

The microcapsules 100 may be utilized in order to prevent wicking, or migration, of the indicator material 120 prior to subjection to an activation action even when the indicator material 120 encapsulated in the microcapsules is exposed to the predetermined environmental stimulus. Alternatively, the microcapsule 100 may insulate the environmental indicator from the predetermined environmental stimulus.

According to some embodiments, the predetermined environmental exposure may be one of a temperature excursion above a predetermined temperature, a temperature excursion above a predetermined temperature threshold for at least a predetermined amount of time, a temperature excursion below a predetermined temperature, a temperature excursion below a predetermined temperature for at least a predetermined amount of time, cumulative exposure to temperature over a time period above a predetermined threshold for at least a predetermined amount of time, an exposure to a particular chemical, an oxygen exposure, an ammonia exposure, an exposure to a particular chemical above a threshold concentration, an exposure to a particular chemical above the threshold concentration for at least a predetermined amount of time, an exposure to at least a predetermined amount of radiation of a particular type, an predetermined electromagnetic exposure, a humidity exposure, an exposure to a humidity level above a predetermined threshold, and an exposure to a humidity level above a predetermined threshold for at least a predetermined amount of time, combinations thereof, and the like.

In one embodiment, the carrier material is a liquifiable solid configured to liquefy in response to a temperature above a predetermined threshold. In another embodiment, the carrier material is a gel configured to, in response to a predetermined environmental exposure above a predetermined threshold, change viscosity such that the gel is substantially liquefied and is capable of fluid flow. For example, the material may be a side-chain crystallizable polymer combined with an alkane wax. Some side-chain crystallizable (SCC) polymers useful in the practice of the present disclosure, alone or in combination, and methods that can be employed for preparing them, are described in O'Leary et al. “Copolymers of poly(n-alkyl acrylates): synthesis, characterization, and monomer reactivity ratios” in Polymer 2004 45 pp 6575-6585 (“O'Leary et al.” herein), and in Greenberg et al. “Side Chain Crystallization of n-Alkyl Polymethacrylates and Polyacrylates” J. Am. Chem. Soc., 1954, 76 (24), pp. 6280-6285 (“Greenberg et al.” herein). The disclosure of each of O'Leary et al. and Greenberg et al. is incorporated by reference herein for all purposes.

Side-chain crystallizable polymers, sometimes called “comb-like” polymers, are well-known and available commercially. These polymers are reviewed in J. Polymer Sci. Macromol. Rev. 8:117-253 (1974), the disclosure of which is hereby incorporated by reference. In general, these polymers contain monomer units X of the formula:

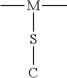

where M is a backbone atom, S is a spacer unit and C is a crystallizable group. These polymers have a heat of fusion (ΔHf) of at least about 20 Joules/g, preferably at least about 40 Joules/g. The polymers will contain about 50 to 100 percent monomer units represented by “X”. If the polymer contains less than 100 percent X, it will in addition contain monomer units which may be represented by “Y” or “Z”, or both, wherein Y is any polar or nonpolar monomer or mixture of polar or nonpolar monomers capable of polymerizing with X and/or Z, and wherein Z is a polar monomer or mixture of polar monomers. Polar groups, e.g., polyoxyalkylenes, acrylates including hydroxyethylacrylate, acrylamides including methacrylamide-will typically increase adhesion to most substrates. If the polar species “Z” is acrylic acid, it is preferred that it comprise about 1-10 wt. percent of the polymer.

The backbone of the polymer (defined by “M”) may be any organic structure (aliphatic or aromatic hydrocarbon, ester, ether, amide, etc.) or an inorganic structure (sulfide, phosphazine, silicone, etc.), and may include spacer linkages which can be any suitable organic or inorganic unit, for example ester, amide, hydrocarbon, phenyl, ether, or ionic salt (e.g., a carboxyl-alkyl ammonium or sulphonium or phosphonium ion pair or other known ionic salt pair).

The side-chain (defined by ‘S’ and ‘C’) may be aliphatic or aromatic or a combination of aliphatic and aromatic, but must be capable of entering into a crystal line state. Common examples are: linear aliphatic side chains of at least 10 carbon atoms, e.g., C4-C22 acryl ates or methacrylates, acrylamides or methacrylanides, vinyl ethers or esters, siloxanes or alpha olefins; fluorinated aliphatic side-chains of at least 6 carbons; and p-alkyl styrene side-chains wherein the alkyl is of 8 to 24 carbon atoms.

The length of the side-chain moiety is usually greater than 5 times the distance between side-chains in the case of acrylates, methacrylates, vinyl esters, acrylamides, methacrylamides, vinyl ethers and alpha olefins. In the extreme case of a fluoroacrylate alternate copolymer with butadiene, the side-chain can be as little as two times the length as the distance between the branches.

In any case, the side-chain units should make up greater than 50 percent of the volume of the polymer, preferably greater than 65 percent of the volume. Specific examples of side-chain crystallizable monomers are the acrylate, fluoroacrylate, methacrylate and vinyl ester polymers described in J. Poly. Sci 10:3347 (1972); J. Poly. Sci 10:1657 (1972); J. Poly. Sci 9:3367 (1971); J. Poly. Sci 9:3349 (1971); J. Poly. Sci. 9:1835 (1971); J.A.C.S. 76:6280 (1954); J. Poly, Sci 7:3053 (1969); Polymer J. 17:991 (1985), corresponding acryl amides, substituted acrylamide and malcimide polymers (J. Poly. Sci: Poly. Physics Ed. 18:2197 (1980)); polyal phaolefin polymers such as those described in J. Poly. 5,156,911 7 Sci. Macromol. Rey, 8:117-253 (1974) and Macromolecules 13:12 (1980), polyalkylvinylethers, polyalkylethylene oxides such as those described in Macromolecules 13:15 (1980), alkylphosphazene polymers, polyamino acids such as those described in Poly. Sci. USSR 21:241, Macromolecules 18:2141, polyisocyanates such as those described in Macromolecules 12:94 (1979), polyurethanes made by reacting amine- or alcohol-containing monomers with long-chain alkyl isocyanates, polyesters and polyethers, polysiloxanes and polysilanes such as those described in Macromolecules 19:611 (1986), and p-alkylstyrene polymers such as those described in J.A.C.S. 75:3326 (1953) and J. Poly. Sci 60:19 (1962). Of specific utility are polymers which are both relatively polar and capable of crystallization, but wherein the crystallizing portion is not affected by moisture. For example, incorporation of polyoxyethylene, polyoxy propylene, polyoxybutylene or copolyoxyalkylene units in the polymer will make the polymer more polar.

In a particularly preferred embodiment herein, in the above structure, —C is selected from the group consisting of —(CH2)—CH3 and —(CF2)n—CF2H, where n is an integer in the range of 8 to 20 inclusive, —S— is selected from the group consisting of —O—, —CH2—, —(CO)—, —O(CO)— and —NR— where R is hydrogen or lower alkyl (1-6C), and —M— is —[(CH2)m —CH]— where m is 0 to 2.

Typical “Y” units include linear or branched alkyl or aryl acrylates or methacrylates, alpha olefins, linear or branched alkyl vinyl ether or vinyl esters, maleicesters or itaconic acid esters, acrylamides, styrenes or substituted styrenes, acrylic acid, methacrylic acid and hydrophilic monomers as detailed in WO84/0387, cited supra.

Some useful side-chain crystallizable polymers, and monomers for preparing side-chain crystallizable polymers, are also available from commercial suppliers, for example, Scientific Polymer Products, Inc., Ontario, N. Y., Sigma-Aldrich, Saint Louis, Mo., TCI America, Portland Oreg., Monomer-Polymer & Dajac Labs, Inc., Trevose, Pa., San Esters Corp., New York, N. Y., Sartomer USA, LLC, Exton Pa., and Polysciences, Inc. Other materials may be SCCs alone, without SCCs, or alkane waxes blended without SCCs.

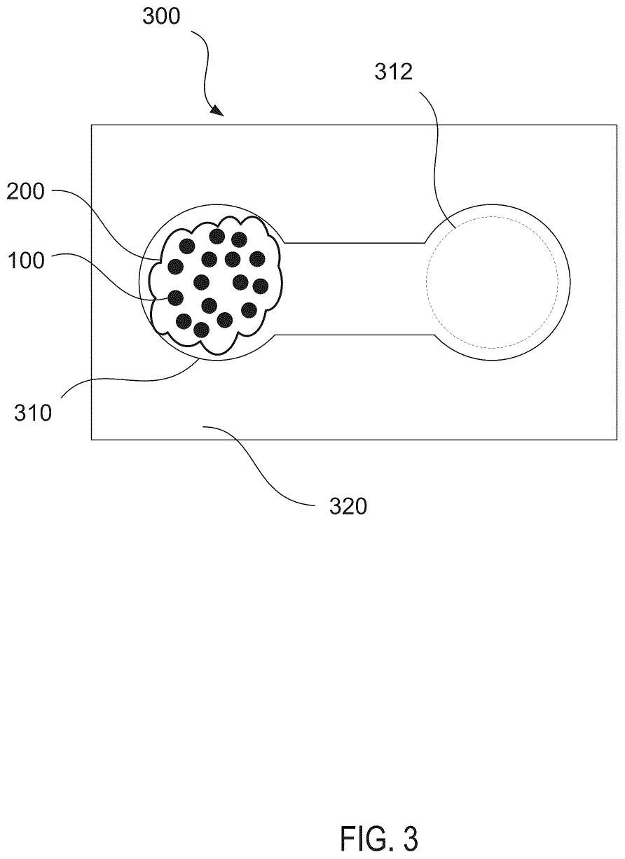

According to some embodiments of the present disclosure, the indicative material of the indicator material 120 may be an additive to the liquefiable carrier material, such that the liquefaction of the indicator material 120, or the movement of liquefied indicator material 120 is made apparent to an observer. In some examples, the indicative material is a dye, or chemical pigment, such as a leuco dye, or other plurality of colored particles carried by the carrier material. In some examples the indicative material is a flash material, which gives off a bright appearance when illuminated with light of a specified wavelength. In some examples, the indicative material is a plurality of conductive particles, or conductive material. In some examples, the indicative material may be one or more chemical reactants, configured to produce a color-state changing chemical reaction when exposed to another chemical reactant. In some such examples, a first of the chemical reactants may be disposed in the indicator material 120 and a second of the chemical reactants may be disposed in a receiving location, (e.g., indicator region 312, Sec. FIGS. 3-6), such that when the indicator material 120 reaches the receiving location the first and second chemical reactants react, producing a color state change. In other such examples, the first of the chemical reactants is disposed in a first plurality of microcapsules, and the second of the chemical reactants is disposed in a second plurality of microcapsules, and the first and second chemical reactants produce the color state changing reaction when the first and second pluralities of microcapsules are activated.

According to some embodiments, the activatable shell 110 is configured to contain the indicator material 120 when the indicator material is in the liquid phase and when the indicator material 120 is in the solid phase. The activatable shell 110 is configured to rupture responsive to the application of an activation action. When the activatable shell 110 is ruptured, the indicator material 120 is released.

The microcapsule 100 may be any size, but in one such embodiment, has an outer diameter length between 50 to 750 μm. The shell 110 may be any size smaller than or equal to the outer diameter of the microcapsule 100, but in one such embodiment, has an outer diameter length between 5 to 25 micrometers (μm). The payload, or the ratio of the total weight of the contents (e.g. indicator material 120) within the microcapsule 100 to the entire weight microcapsule 100 including the contents contained within the microcapsule 100, can range from 50 percent to 90 percent. It will be appreciated that a variety of microcapsule shell 110 materials may be chosen, depending on the application, the type of activation, and the nature of the contents of the microcapsule 100. In general, the microcapsules 100 should resist the passage, whether by flow, diffusion, or migration, of the contents of the microcapsule 100 prior to activation.

For example, the shell 110 may be formed in whole or in part by a wax, e.g., an alkane wax, or other acid resistant compound having a relatively high melting point, e.g., fatty acid amide, an ester or Elvax EVA resin. For example, the melting point may be in a range of about 50 degrees Celsius (C) to about 300 degrees C., from about 100 degrees C. to about 300 degrees C., from about 150 degrees C. to about 300 degrees C., from about 200 degrees C. to about 300 degrees C., from about 250 degrees C. to about 300 degrees C. Generally, the shell should have a higher melting point than the maximum temperature the microcapsule is expected to be exposed to in normal use, to prevent it from rupturing or melting prematurely.

In another example, the shell 110 may be formed in whole or in part by a polymer coating having a high glass transition temperature (T_{g}) e.g. Polysulfonc. For example, the glass transition temperature may be in a range of about 50 degrees C. to about 300 degrees C., from about 100 degrees C. to about 300 degrees C., from about 150 degrees C. to about 300 degrees C., from about 200 degrees C. to about 300 degrees C., from about 250 degrees C. to about 300 degrees C. For example, Polysulfone, with a T_{g} of about 190 C may be used. In additional examples, the microcapsules 104 may be one of Styrene Maleic Anhydride (SMA), Polyphenylene Ether (PPE), Cellulose Acetate, Cellulose Diacetate, Polyacrylate, Polyamide, Polycarbonate, polyether ether ketone, Polyether Sulfone, PET, PFA, polymethyl methacrylate (PMMA) or Polyimide.

In another example, the shell 110 may be formed in whole or in part by a low molecular weight polymer gel having a high melting point, e.g., fatty acid amide, an ester or Elvax EVA resin. For example, the melting point may be in a range of about 100 degrees C. to about 300 degrees C., from about 150 degrees C. to about 300 degrees C., from about 200 degrees C. to about 300 degrees C., from about 250 degrees C. to about 300 degrees C. Additionally, in some examples, the polymer gel has a molecular weight in a range from about 1 grams per mole (g/mol) to 100,000 g/mol, from about 3,500 g/mol to 6,000 g/mol and from about 200 g/mol to 2,000 g/mol.

Alternatively, the shell 110 may be formed in whole or in part by a gel, gelatin, protein, polyurea formaldehyde, polymelamine formaldehyde, wax material, melamine, or an emulsion. The microcapsules may be available in wet and dry formulations. Polymelamine and polyurca formaldehyde can both be used for encapsulations via interfacial polymerization, which uses two immiscible phases. Once separated in the same vessel, a reaction is initiated at the interface of the two immiscible phases in the presence of an initiator and the material to be encapsulated. As polymerization occurs, microcapsules form around the core material. The microcapsule 100 releases the contents of the microcapsule 100 upon rupturing.

The microcapsule 100 is initially in an unactivated form, capable of being configured to transition to an activated form when activated through exposure to an activation action, e.g., the application of heat, pressure, and/or heat and pressure exceeding a predetermined threshold. In the unactivated form, the shell 110 of the microcapsule 100 maintains separation between the contents of the microcapsule 100 and any external environmental stimuli and/or contains a phase change of the contents of the microcapsule 100 in response to any external environmental stimuli.

The microcapsule 100 may be “activated” or ruptured by exposing the microcapsule 100 to an activation action (e.g. activation stress, activation exposure, activation event, etc.) exceeding a predetermined activation threshold. The activation action may cause the microcapsule 100 to fracture, melt, break, dissolve, sublime, become porous, or otherwise disengage, allowing the release of the contents of the microcapsule 100.

According to some embodiments, the activation action may be an application of at least one of an activation heat and an activation pressure. In some examples, the temperature threshold for activation may be from about 0 degrees C. to 300 degrees C., from about 90 degrees C. to 115 degrees C., from about 100 degrees C. to 200 degrees C., from about 100 degrees C. to 300 degrees C., and from about 200 degrees C. to 300 degrees C.

In some examples, where the activation action is a thermal stress, the temperature threshold for activation may be a temperature exceeding about 35 degrees C., a temperature exceeding about 40 degrees C., a temperature exceeding about 45 degrees C., a temperature exceeding about 50 degrees C., a temperature exceeding about 55 degrees C., a temperature exceeding about 60 degrees C., a temperature exceeding about 65 degrees C., a temperature exceeding about 70 degrees C., a temperature exceeding about 75 degrees C., a temperature exceeding about 80 degrees C., a temperature exceeding about 85 degrees C., a temperature exceeding about 90 degrees C., a temperature exceeding about 95 degrees C., and a temperature exceeding about 100 degrees C. It will be appreciated that the activation heat ranges given are purely exemplary and the microcapsules 100 can be formed to respond to other temperature ranges.

Activation may be achieved by applying a high temperature for a very short interval, e.g., a few milliseconds. For example, the mass or heat of fusion of the indicator may be much greater than the mass or heat of fusion of a barrier that needs to be removed, allowing a short exposure to high temperature to remove or alter the microcapsule 100 without significantly affecting the contents of the microcapsule 100.

In some cases, pressure may also contribute to the activation, e.g., by breaking microcapsules 104, either alone or in combination with elevated temperature. In such embodiments, the activation action is a compressive stress, or a shearing stress, where the predetermined activation threshold is a stress exceeding about 0.1 pounds per square inch (psi), a stress exceeding about 0.5 psi, a stress exceeding about 1 psi, a stress exceeding about 2 psi, a stress exceeding about 5 psi, a stress exceeding about 10 psi, or a stress exceeding about 15 psi.

The activation action may include the application of heat to reach an activation temperature, the application of an activation pressure, or a combination thereof (e.g., by a thermal printhead). In some examples, the temperature threshold for activation may be from about −40° C. to 100° C., from about 5° C. to 35° C., from about 0° C. to 300° C., from about 90° C. to 110° C., from about 100° C. to 200° C., from about 100° C. to 300° C., and from about 200° C. to 300° C. Activation may be achieved by applying a high temperature for a very short interval, e.g., a few milliseconds. In this manner, even if the temperature needed to activate the device exceeds the temperature that a temperature exposure indicator is configured to indicate, the exposure may be so short that the indicator itself is not affected. For example, the mass or heat of fusion of the indicator may be much greater than the mass or heat of fusion of a barrier that needs to be removed, allowing a short exposure to high temperature to remove or alter the microcapsule 100 without significantly affecting the contents of the microcapsule 100. Typical thermal print heads have temperatures in the range from about 100° C. to 300° C., which may be tuned downward for select applications to from about 100° C. to 200° C. They are typically exposed to the thermal print heads for a brief period of time, for example a few milliseconds. The microcapsule 100 itself responds when it reaches a temperature of in a range from about −40° C. to 100° C., from about 5° C. to 35° C., from about 0° C. to 300° C., from about 90° C. to 110° C., from about 100° C. to 200° C., from about 100° C. to 300° C., and from about 200° C. to 300° C. It will be appreciated that the activation temperature ranges given are purely exemplary and other ranges may be sufficient to activate the microcapsules 100, where such pressure ranges may vary based on a composition of the shell 110, a thickness of the shell 110, a ratio between the shell thickness or weight to volume or weight of the indicator material, a diameter of the microcapsules, a temperature applied to the shells, etc. In some cases, pressure may also contribute to the activation, e.g., by breaking microcapsules 100, either alone like an impact printer, or in combination with elevated temperature. In some examples, the activation pressure required to activate the microcapsules 100 may be from about 1.5 to 8 pounds per square inch or from about 4 to 15 pounds per square inch. It will be appreciated that the activation pressure ranges given are purely exemplary and other ranges may be sufficient to activate the microcapsules 100, where such pressure ranges may vary based on a composition of the shell 110, a thickness of the shell 110, a ratio between the shell thickness or weight to volume or weight of the indicator material, a diameter of the microcapsules, a temperature applied to the shells, etc.

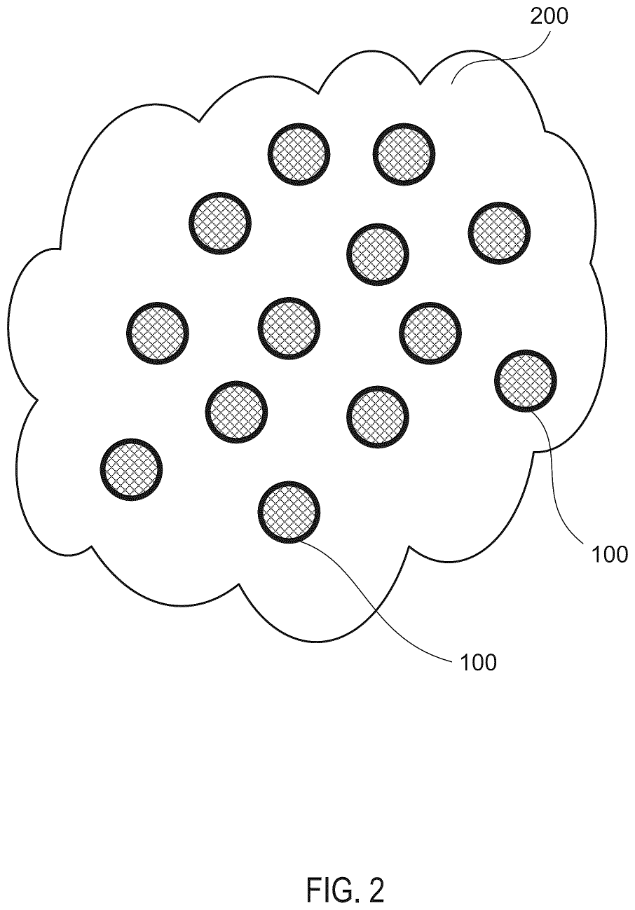

FIG. 2 illustrates a bonding material 200, in which a plurality of microcapsules 100 are disposed, according to embodiments of the present disclosure. The bonding material 200 may be a polymer having side-chain crystallinity, polymeric materials, an alkane, a wax, an alkane wax, an ester, and/or combinations thereof. As a non-limiting example, the bonding material 200 may be a side-chain crystallizable polymer combined with an alkane wax, as described with reference to the indicator material of FIG. 1. The bonding material 200 is configured to have a liquefaction point higher than the liquefaction point of the indicator material 120, such that the bonding material is does not liquefy responsive to the same threshold of the predetermined environmental exposure that the indicator material 120 is configured to respond to. The bonding material 200 is configured to be dispensed in a liquid phase, while containing the microcapsules 100, and solidify after dispensation. When the bonding material 200 solidifies, the microcapsules 100 are suspended, embedded, or otherwise contained in the bonding material 200, such that the microcapsules 100 are not able to move within the solidified bonding material 200.

In some examples the liquefaction point of the bonding material 200 may be defined within a range bounded by 40 degrees C. and 115 degrees C. as compared to the response temperature of the indicator material 120 which may be defined within a range bounded by −15 degrees C. and 35 degrees C. In some examples, the liquification point of the bonding material 200 may be 25% to 50%, 50% to 100%, 100% to 1,000%, 1,000% to 10,000%, or 25% to 15,000% greater than the response temperature of the indicator material 120. As a non-limiting example, the carrier material of the indicator material 120 within the microcapsules 100 may be one of (i) pentadecane having a melt point or response temperature of 10 degrees C., (ii) a blend of pentadecane having a melt point of 10 degrees C. and a Side Chain Crystalline (SCC) polymer having a melt point of 10 degrees C. to provide a response temperature of 10 degrees C., (iii) a blend of dodecane having a melt point of −10 degrees C. and undecane having a melt point of −26 degrees C. providing a response temperature of a −15 degrees C., (iv) octadecane having a melt point or response temperature between 28 degrees C. and 30 degrees C., or other suitable materials or blends of materials to provide a response temperature of between −15 degrees C. and 35 degrees C. As a non-limiting example, the bonding material may be (i) heneicosane having a melt point or liquification point of 40 degrees C., (ii) heptatriacontan having a melt point or liquification point of 80 degrees C., (iii) hectane having a melt point or liquification point of 115 degrees C., or other suitable materials or blends of materials to provide a response temperature of between 40 degrees C. and 115 degrees C. As described herein, utilizing a bonding material that has liquification point that is well above that of the carrier material of the indicator material allows the carrier material to flow to the wicking material when it is released by rupturing the encapsulant shell and is exposed to a response temperature instead of mixing with the bonding material 200 which remains in its solid form at and well of above the response temperature.

FIGS. 3 illustrates an indicator 300, which may serve as a platform for the several examples of environmentally sensitive indicators disclosed herein, according to embodiments of the present disclosure. FIGS. 3-6 illustrate various states of the indicator 300, according to embodiments of the present disclosure. Generally, an indicator 300 includes a wick 310, which may be formed of a wicking material, disposed on a substrate 320. A bonding material 200, containing microcapsules 100, may be disposed proximately to the wick 310. As one example, the bonding material 200 containing the microcapsules 100 on the substrate 320 and the wick can be deposited over the bonding material 200 containing the microcapsules 100 and on the substrate 320. As another example, the wick 310 can be deposited on the substrate 320 and the bonding material 200 including the microcapsules 100 can be deposited on the wick 310. As another example, the bonding material 200 including the microcapsules 100 can be deposited on the substrate 320 adjacent to the wick 310 deposited on the substrate 320. The indicator 300 may further include an indicator region 312, where an observable change may occur when the indicator 300 responds to a predetermined environmental exposure. In FIGS. 3-6, the wick is illustrated as having a “dog bone” shape, however this disclosure contemplates embodiments where the wick 310 has other forms and shapes. In FIGS. 3-6, the indicator region is illustrated as being a portion of the wick, opposite to where the bonding material 200 is disposed, however this disclosure contemplates embodiments where the indicator region has other forms and shapes. Non-limiting examples of contemplated indicator regions include a demarcated portion of a wick, a non-demarcated portion of the wick, the entirety of the wick, an area proximate to the wick, a reservoir or containment volume disposed proximately to the wick, and the like.

In some examples, the wick 310 may be formed from a wicking material, which may include woven polyester, nonwoven polyester, polyamide and blended elastane and polyester, carbon fiber, Teslin synthetic paper, polyethylene, polypropylene, polytetrafluoroethylene, woven nylon, or any material suitable to draw water from a medium by capillary action.

In some examples, the substrate 320 may be made with a paper or polyethylene terephthalate (PET). In other examples, the substrate may be made with any other suitable non-conductive material or any breathable film, such as cloth or plastic (e.g., polyethylene terephthalate (PET), polyvinyl chloride (PVC), polyvinyl acetate (PVAC), etc.).

FIG. 3 illustrates the unactivated state of the indicator 300, according to embodiments of the present disclosure. In the unactivated state, the shells 110 of the microcapsules 100 are intact, and contain the indicator material 120, irrespective of whether the indicator material 120 is liquefied or solidified. The bonding material is in a solid phase, such that the microcapsules 100 are not able to move within the solidified bonding material 200. The bonding material 200 is secured to the wick 310 via adhesion, where the bonding material 200 self-secures to the wick, but does not saturate, fill, or otherwise permeate pores of the wick 310. In the unactivated state, the indicator region 312 is in a first state. The first state indicates that the indicator 300 has not been exposed to the predetermined environmental exposure.

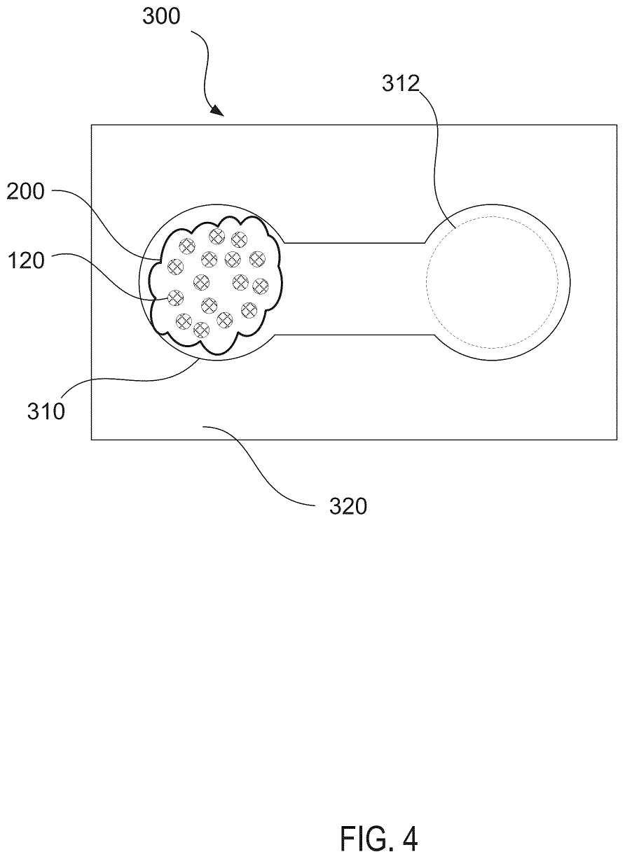

FIG. 4 illustrates an activated and unexposed state of the indicator 300, according to embodiments of the present disclosure. When the indicator 300, or more specifically, the microcapsules 100, are exposed to the activation action, the microcapsules 100 rupture, releasing the indicator material 120. When the microcapsules 100 rupture, the indicator 300 is “activated.” When the indicator 300 is activated, liquefaction of the indicator material 120 responsive to the predetermined environmental exposure causes the migration of the liquefied indicator material 120 into the wick 310. However, in the activated and unexposed state, the indicator 300 has yet to be exposed to the predetermined environmental exposure. Thus, the indicator material 120 is in the solid phase and is not drawn into the wick 310. In the activated and unexposed state, the indicator region 312 is in a first state, as the indicator material 120 is not present in the indicator region 312 to produce the observable effect.

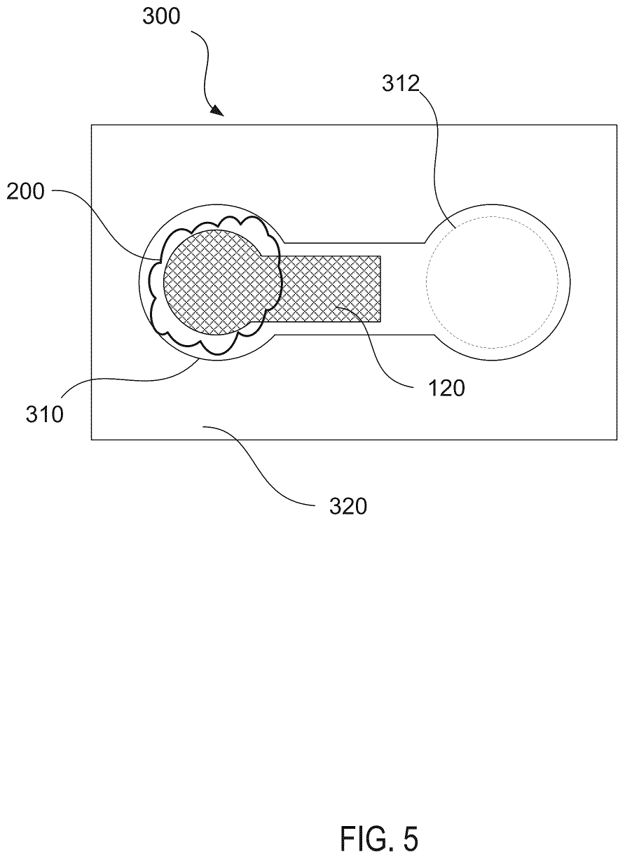

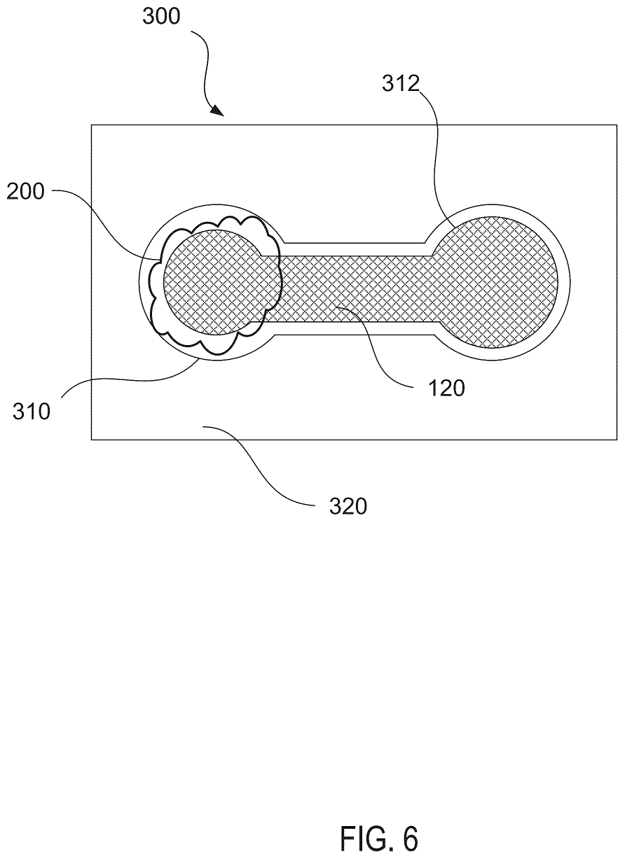

FIG. 5 illustrates an activated and exposed state of the indicator 300, according to embodiments of the present disclosure. After the microcapsules 100 have been activated, the indicator material 120 is released from the microcapsule. When the indicator 300 is subsequently exposed to the predetermined environmental exposure, the indicator material 120 liquefies and migrates into the wick 310. The liquefied indicator material 120 may be drawn into the wick 310 via wicking action or capillary action, and the indicator material may saturate (e.g., partially, or fully), fill, or otherwise permeate pores of the wick. The indicator 300 is configured such that the indicator material 120 migrates along the wick 310, or progressively saturates the wick 310, until the indicator material 120 reaches the indicator region 132 (see FIG. 6). FIG. 5 illustrates a transitional period of the indicator where the indicator material 120 has yet to reach the indicator region 312.

In some examples, the indicator 300 is configured to indicate exposure to a predetermined environmental exposure for a predetermined amount of time. In such embodiments, the indicator material 120 may solidify responsive to a cessation of the predetermined environmental exposure. In such embodiments, the wick 310 may be configured such that the indicator material 120 must be in a liquid state for a predetermined amount of time to fully migrate through the wick 310 and reach the indicator region 312.

In some examples, the indicator material 120 may liquefy and solidify responsive to successive exposures and cessation of the predetermined environmental exposure, and the indicator material 120 reaches the indicator region 312 after a cumulative amount of time in which the indicator is exposed to the predetermined environmental exposure reaches a predetermined threshold. In such examples, the wick 310 may be configured to have a specific length, such that the indicator 300 is time-dependent. A wick 310 of increased length (e.g., an increased distance between the portion of the wick on which the bonding material is disposed and the indicator region 132) may provide for a longer time-dependency, as the indicator material 120 would have a greater distance to travel to reach the indicator region 132. Time-dependency may also be tuned by adjusting the viscosity of the indicator material 120, as a more viscous indictor material 120 may wick slower than a less viscous indicator material 120.

FIG. 6 illustrates an activated and exposed state of the indicator 300, where the indicator material 120 has reached the indicator region 312, according to embodiments of the present disclosure. When the indicator material 120 reached the indicator region 312, the indicator material 120 produces an observable effect in the indicator region 312.

In some examples, the indicator material 120 has a color state that is visibly distinct from the color state of the indicator region 312, such that when the indicator material 120 reaches the indicator region 312 the presence of the indicator material 120 is the observable effect. In such examples, the color state of the indicator material may be dependent upon the indicative material contained in the indicator material 120, which may include a chemical pigment, ink, or dye.

In some examples, the indicator material 120 may reveal or obscure an indicium in the indicator region 312. In such examples, the indicium to be obscured or revealed may have a color state distinct or contrasted from the color state of the indicator material 120, such that the indicium appears to undergo a change of color state when the indicator material 120 migrates into the indicator region 312. In some examples, when the indicum is revealed, the indicum includes a symbol, image, or natural language text.

As a non-limiting illustrative example, the indicator region 132 may include a saturable indium having the form of an “X” (e.g., to indicate exposure to the predetermined environmental exposure) and having a color state that does not visually contrast the substrate 320 such that the “X” is not readily viewable to an observer. The indicator material 120 includes an indicative material having a color state that visually contrasts the substrate 320, such that when the indicator material 120 reaches the indicator region 312, the indicator material 120 saturates the indicum, and the “X” becomes readily viewable to the observer.

In some examples, the indicator region 312 includes a first chemical reactant, configured to react with a second chemical reactant contained in or comprising the indicative material in the indicator material 120, such that when the indicator material 120 reaches the indicator region 312, the second chemical reactant in the indicative material reacts with the first chemical reactant, and produces a chemical product, where the chemical product or the chemical reaction creates an observable effect. In such examples, the chemical product may have a color state that is visibly distinct from the indicator material 120 and the indicator region 312. In some examples, the first chemical reactant forms the indicia, which is initially not visible to an observer, or has a first color state, and becomes visible, or changes color state upon contact with the second chemical reactant contained in the indicator material.

In some examples, the indicator material 120 includes conductive materials which are configured to change an electrical property of the indicator region 312. In such examples, the indicator region 132 may have a first electrical property (e.g., a first measured value of one or more of resistance, capacitance, conductivity, and impedance) when the indicator material 120 is not in the indicator region 312, and have a second electrical property (e.g., a second measured value of one or more of resistance, capacitance, conductivity, and impedance) when the indicator material 120 is in the indicator region 312. The electrical property may be monitored across the indicator region 312 by a circuit, and the change from the first electrical property to the second electrical property when the indicator material 120 reaches the indicator region 132 induces a change in behavior of the circuit, which may be observed by a user. Stated differently, the observable effect produced by the indicator material 120 reaching the indicator region 312 is the change from the first electrical property to the second electrical property. In some examples the conductive materials may include particles containing copper, particles containing silver, particles containing graphite, particles containing conductive metals, particles containing conductive non- metal materials, and combinations thereof.

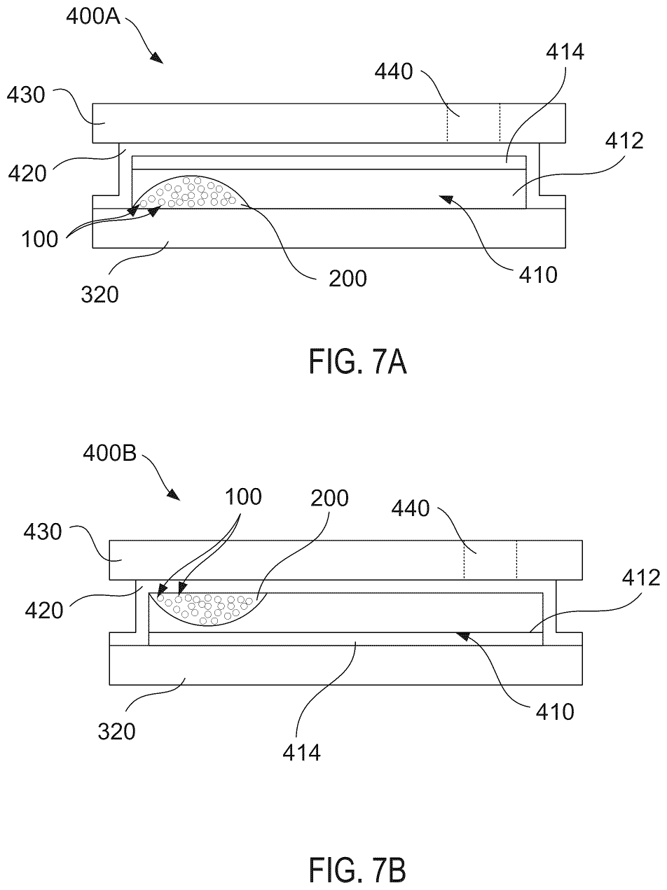

FIGS. 7A-B illustrate indicators 400A and 400B, respectively, which may serve as a platform for the several examples of environmentally sensitive indicators disclosed herein, according to embodiments of the present disclosure. The indicators 400A-B include the substrate 320, a laminated wick 410, the bonding material 200, containing the microcapsules 100, a scaling layer 420, and a cover or top layer 430. The laminated wick 410 may be formed of a wicking material 412 (e.g., forming one layer of the laminated wick 410) and a laminate material 414 (c.g., forming another layer of the laminated wick 410). The wicking material 412 may include woven polyester, nonwoven polyester, polyamide and blended elastane and polyester, carbon fiber, Teslin synthetic paper, polyethylene, polypropylene, polytetrafluoroethylene, woven nylon, or any material suitable to draw water from a medium by capillary action. As a non-limiting example, the laminate material 414 may be a transparent polyester film.

The bonding material 200, containing microcapsules 100, may be disposed proximately to the wicking material of the laminated wick 410. As one example, the bonding material 200 containing the microcapsules 100 on the substrate 320 and the laminated wick 410 may be deposited over the bonding material 200 containing the microcapsules 100 and on the substrate 320, as shown if FIG. 7A, so that the wicking material 412 is in contact with the bonding material 200. As another example, the laminated wick 410 may be deposited on the substrate 320, as shown in FIG. 7B, so that the laminated material 414 of the laminated wick 410 is adjacent to the substrate 320 and the bonding material 200 including the microcapsules 100 may be deposited on the wicking material 412 of the laminated wick 410. As another example, the bonding material 200 including the microcapsules 100 may be deposited on the substrate 320 adjacent to the wicking material 412 of the laminated wick 410 deposited on the substrate 320. As a non-limiting example, the sealing layer 420 may be a transparent polyester film. In one example, the sealing layer 420 and the laminated material 414 of the laminated wick 410 may be formed from the same materials. In one example, the sealing layer 420 and the laminated material 414 of the laminated wick 410 may be formed from different materials. The sealing layer 420 may be adhered to the substrate 320 around a perimeter of the laminated wick 410. The cover 430 may be disposed on the scaling layer 420. The cover may be transparent or opaque. In one example, the cover 430 may be formed from the same material as the substrate 320. For embodiments in which the cover is opaque, the cover may include a viewing window or opening 432 through which an indicator region 440 may be viewable. An observable change may occur in the indicator region when the indicator 400 responds to a predetermined environmental exposure (e.g., after the indicator material 120 is released from the microcapsules and migrates along the wicking material 412 in response to the predetermined environmental exposure).

In some examples, the indicators 400A-B may be devoid of the bonding material 200 such that the microcapsules 100 may be deposited without the bonding material and/or may be devoid of the microcapsules 100 such that the indicator material 120 may be deposited without encapsulation.

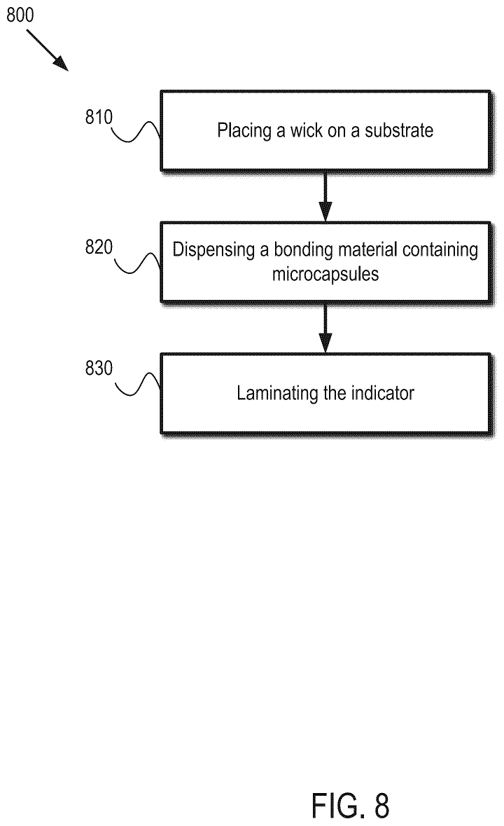

FIG. 8 illustrates a flowchart describing a method 800 for producing an indicator (e.g., indicators 300, 400A-B), according to embodiments of the present disclosure.

Block 820 of the method 800 describes placing a wick (e.g., wick 310, 410) on a substrate (e.g., substrate 320), according to embodiments of the present disclosure. In some examples, the wick may be formed from a wicking material, which may include woven polyester, nonwoven polyester, polyamide and blended elastane and polyester, carbon fiber, Teslin synthetic paper, polyethylene, polypropylene, polytetrafluoroethylene, woven nylon, or any material suitable to draw water from a medium by capillary action. In some examples, the substrate may be made with a paper or polyethylene terephthalate (PET). In other examples, the substrate may be made with any other suitable non-conductive material or any breathable film, such as cloth or plastic (e.g., polyethylene terephthalate (PET), polyvinyl chloride (PVC), polyvinyl acetate (PVAC), etc.). In some examples, the wick comprises a laminate layer, such as a polyester joined laminated to the wicking material and placing the wick may include cutting a laminated sheet of the laminate layer and the wicking material into a wicks (e.g., using a die cutter), picking one of the wicks using a pick and place device, and placing the selected wick onto the substrate using the pick and place device.

In some examples, the wick may be secured to the substrate with an adhesive. In some such embodiments, the adhesive, or an adhesive layer may be previously disposed on the substrate, or previously disposed on the wick. In other such examples, the adhesive is dispensed onto the substrate or wick as the wick is installed on the substrate.

In some examples, the substrate may include a demarcation or an indentation corresponding to a placement location of the wick.

Block 820 of the method 800 describes dispensing a bonding material (e.g., bonding material 200) containing microcapsules (e.g., microcapsules 100) proximately to the wick, according to embodiments of the present disclosure. In some examples, the microcapsules contain an indicator material (e.g., indicator material 120) microencapsulated in an activatable shell (e.g., shell 110). The bonding material is dispensed in a liquid state at a dispensation temperature which is marginally above the liquefaction point (and the solidification point) of the bonding material, such that when the bonding material contacts the wick, the bonding material solidifies before the bonding material permeates pores of the wick. In some examples the dispensation temperature is less than 10 degrees C. above the solidification point of the bonding material, preferably less than 5 degrees C. above the solidification point of the bonding material, and more preferably less than 2 degrees C. above the solidification point of the bonding material.

In some examples, the bonding material is dispensed such that the bonding material abuts or is adjacent to the wick. In some examples, the bonding material is dispensed atop the wick. In some examples, the substrate includes an indentation or similar structure, configured to serve as reservoir for the bonding material.

In embodiments where the indicator employs an indicator material configured to respond to a thermal predetermined environmental exposure (e.g., a temperature excursion above a predetermined temperature, a temperature excursion above a predetermined temperature threshold for at least a predetermined amount of time, a cumulative exposure to temperature over a time period above a predetermined threshold for at least a predetermined amount of time), the dispensation temperature is greater than the response temperature of the indicator material, which ensures that the bonding material does not liquefy responsive to the predetermined environmental exposure to which the indicator material liquefies, which could interfere with the proper operation of the indicator and negatively impact function thereof. In such embodiments, the activatable shells of the microcapsules contain the indicator material when the indicator material is in the liquid state, such that the liquefied indicator material is prevented from migrating into the wick prior to the activatable shells being activated.

Block 830 of the method 700 describes laminating the indicator, according to embodiments of the present disclosure. After the bonding material containing the microcapsules is dispensed onto the wick, the indicator is ready to be activated. In some examples, the indicator is further laminated with a sealing layer to protect the indicator during further processing (e.g., packaging and transit) before the indicator is activated for use by an end user.

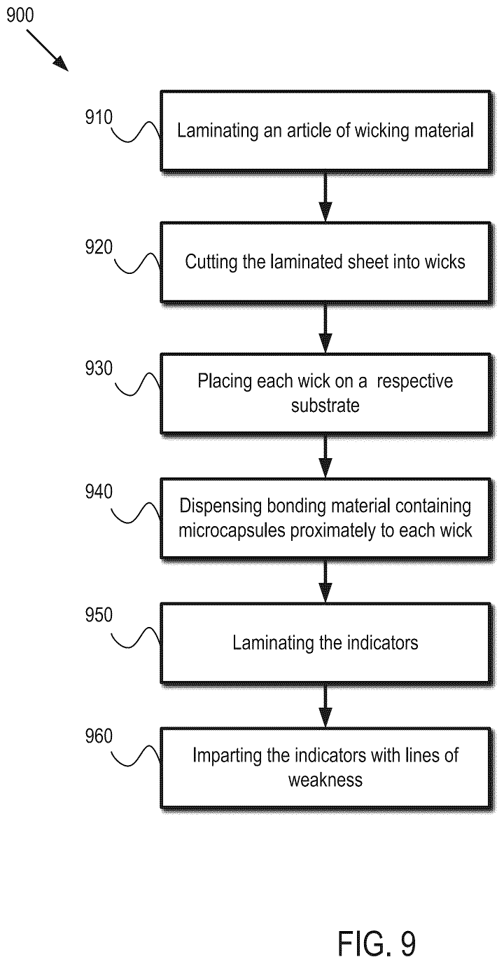

FIG. 9 illustrates a flowchart for a method 900 of forming a plurality of indicators, according to embodiments of the present disclosure. Block 910 of method 900 describes laminating an article of wicking material to form a laminated sheet (e.g., forming an embodiment of laminated wick 410). In some examples, wicking materials are fragile due to porosity, and may be prone to disintegration without additional support. Laminating the wicking material provides a structural backing to the wicking material, which may increase the structural integrity of the wicking material. In some examples, the wicking material is laminated with an adhesive layer, which may aid in retaining the position of fibers within the wicking material. In some examples, the wicking material has sufficient structural integrity to forego lamination, and block 910 may be omitted from the method 900.

Block 920 of method 900 describes cutting the laminated sheet into individual wicks, according to embodiments of the present disclosure. Wicks (e.g., wick 410) may be cut, punched or otherwise separated from the laminated sheet, such that individual wicks are formed. The laminated layer may be retained in each wick to increase the structural integrity of each wick.

Block 930 of the method 900 describes placing each wick (e.g., wick 410) on a respective substrate (e.g., substrate 320), according to embodiments of the present disclosure. In some examples, the substrates are separated, and each wick is placed on a respective separate substrate. In other examples, the substrates are elements of a sheet or article of substrate material, where portions of the substrate are demarcated (e.g., physically, or digitally) into individual substrates to be separated at a later time.

In some examples, the article of substrate material is a roll, and wick placement occurs in a continuous process, where demarcated sections of substrate are successively unwound from the roll of substrate material such that wicks may be placed on the successively unwound substrates.

In some examples, the wicks may be secured to the substrates with an adhesive. In some such embodiments, the adhesive, or an adhesive layer may be previously disposed on the substrate, or previously disposed on the wick. In other such examples, the adhesive is dispensed onto the substrate or wick as the wick is installed on the substrate.

In some examples, the substrate may include a demarcation or an indentation corresponding to a placement location of the wick.

Block 940 of the method 900 describes dispensing a bonding material (e.g., bonding material 200) containing microcapsules (e.g., microcapsules 100) onto the wick, according to embodiments of the present disclosure. In some examples, the microcapsules contain an indicator material (e.g., indicator material 120) microencapsulated in an activatable shell (e.g., shell 110). The bonding material is dispensed in a liquid state at a dispensation temperature which is marginally above the liquefaction point (and the solidification point) of the bonding material, such that when the bonding material contacts the wick, the bonding material solidifies before the bonding material permeates pores of the wick. In some examples the dispensation temperature is less than 10 degrees C. above the solidification point of the bonding material, preferably less than 5 degrees C. above the solidification point of the bonding material, and more preferably less than 2 degrees C. above the solidification point of the bonding material.

In some examples, the bonding material is dispensed such that the bonding material abuts or is adjacent to the wick. In some examples, the bonding material is dispensed atop the wick. In some examples, the substrate includes an indentation or similar structure, configured to serve as reservoir for the bonding material.

In embodiments where the indicators employ an indicator material configured to respond to a thermal predetermined environmental exposure (e.g., a temperature excursion above a predetermined temperature, a temperature excursion above a predetermined temperature threshold for at least a predetermined amount of time, a cumulative exposure to temperature over a time period above a predetermined threshold for at least a predetermined amount of time), the dispensation temperature is greater than the response temperature of the indicator material, which ensures that the bonding material does not liquefy responsive to the predetermined environmental exposure to which the indicator material liquefies, which could interfere with the proper operation of the indicator In such embodiments, the activatable shells of the microcapsules contain the indicator material when the indicator material is in the liquid state, such that the liquefied indicator material is prevented from migrating into the wick prior to the activatable shells being activated.

Block 950 of the method 900 describes laminating the indicators, according to embodiments of the present disclosure. After the bonding material containing the microcapsules is dispensed onto the wicks, the indicators are ready to be activated. In some examples, the indicator is further laminated with a sealing layer to protect the indicator during further processing (e.g., packaging and transit) before the indicator is activated for use by an end user.

Block 960 of the method 900 describes imparting the indicator with lines of weakness, according to embodiments of the present disclosure. In examples where the substrates are not separated prior to wicks being placed thereon, the article of substrate material on which the indicators are constructed may be imparted with lines of weakness to facilitate the separation of individual indicators from one another. In some examples, the lines of weakness are imparted through the sealing layer. In some examples, imparting lines of weakness may include scoring, perforating, embossing indentations, laser etching, or partial cutting. In some examples, the indicators may be separably cut from one another.

Following the imparting the indicators with lines of weakness, in some examples where the article of substrate material is a roll, the indicators may be wound onto a spool or roll of activatable indicators.

In the foregoing specification, specific embodiments have been described. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of present teachings. Additionally, the described embodiments/examples/implementations should not be interpreted as mutually exclusive, and should instead be understood as potentially combinable if such combinations are permissive in any way. In other words, any feature disclosed in any of the aforementioned embodiments/examples/implementations may be included in any of the other aforementioned embodiments/examples/implementations.

The benefits, advantages, solutions to problems, and any element(s) that may cause any benefit, advantage, or solution to occur or become more pronounced are not to be construed as a critical, required, or essential features or elements of any or all the claims. The claimed invention is defined solely by the appended claims including any amendments made during the pendency of this application and all equivalents of those claims as issued.

Moreover in this document, relational terms such as first and second, top and bottom, and the like may be used solely to distinguish one entity or action from another entity or action without necessarily requiring or implying any actual such relationship or order between such entities or actions. The terms “comprises,” “comprising,” “has”, “having,” “includes”, “including,” “contains”, “containing” or any other variation thereof, are intended to cover a non- exclusive inclusion, such that a process, method, article, or apparatus that comprises, has, includes, contains a list of elements does not include only those elements but may include other elements not expressly listed or inherent to such process, method, article, or apparatus. An element proceeded by “comprises . . . a”, “has . . . a”, “includes . . . a”, “contains . . . a” does not, without more constraints, preclude the existence of additional identical elements in the process, method, article, or apparatus that comprises, has, includes, contains the element. The terms “a” and “an” are defined as one or more unless explicitly stated otherwise herein. The terms “substantially”, “essentially”, “approximately”, “about” or any other version thereof, are defined as being close to as understood by one of ordinary skill in the art, and in one non-limiting embodiment the term is defined to be within 10%, in another embodiment within 5%, in another embodiment within 1% and in another embodiment within 0.5%. The term “coupled” as used herein is defined as connected, although not necessarily directly and not necessarily mechanically. A device or structure that is “configured” in a certain manner is configured in at least that manner, but may also be configured other manners that are not listed.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter may lie in less than all features of a single disclosed embodiment. Thus, the following claims are hereby incorporated into the Detailed Description, with cach claim standing on its own as a separately claimed subject matter.

1. An activatable temperature exposure indicator having a response temperature, comprising:
a substrate;
a wick physically coupled to or contained in the substrate;
an indicator region, on or adjacent to a first end of the wick; and
a bonding material containing a plurality of microcapsules disposed on or adjacent to a second end of the wick, opposite the first end;
wherein the bonding material has a liquefaction temperature higher than the response temperature,
wherein each microcapsule of the plurality of microcapsules contains an indicator material microencapsulated in an activatable shell,
wherein the indicator material is configured to liquefy responsive to exposure to a temperature at or above the response temperature,
wherein the activatable shells are configured to contain the indicator material when liquefied,
wherein the activatable shells are configured to rupture in response to an application of an activation action exceeding a predetermined activation threshold, releasing the indicator material,
wherein the indicator material is configured to permeate pores of the wick and migrate along the wick into the indicator region when liquefied and released from the microcapsules, producing an observable change in the indicator region,
wherein the bonding material couples the microcapsules to the wick without filling pores of the wick, and
wherein the bonding material facilitates migration of liquefied indicator material into the wick, and blocks migration of the microcapsules into the wick.

2. The activatable temperature exposure indicator of claim 1, wherein the wick and the bonding material containing the microcapsules are laminated between the substrate and a sealing layer.

3-6. (canceled)

7. The activatable temperature exposure indicator of claim 1, wherein the observable change is a change in a color state selected from a group consisting of: a change of reflectivity, a change in transparency, a change in hue, a change in chroma, a change in apparent color, and combinations thereof.

8. The activatable temperature exposure indicator of claim 1, wherein the observable change is a change in an electrical property selected from a group consisting of: a change in conductivity, a change in resistance, a change in impedance, a change in capacitance, and combinations thereof.

9. The activatable temperature exposure indicator of claim 1, wherein the activation action is a compressive stress and the predetermined activation threshold is selected from a group consisting of: a stress exceeding 0.1 psi a stress exceeding 0.5 psi, a stress exceeding 1 psi, a stress exceeding 2 psi, a stress exceeding 5 psi, a stress exceeding 10 psi, and a stress exceeding 15 psi.

10. The activatable temperature exposure indicator of claim 1, wherein the activation action is a shear stress and the predetermined activation threshold is selected from a group consisting of: a stress exceeding 0.1 psi a stress exceeding 0.5 psi, a stress exceeding 1 psi, a stress exceeding 2 psi, a stress exceeding 5 psi, a stress exceeding 10 psi, and a stress exceeding 15 psi.

11. The activatable temperature exposure indicator of claim 1, wherein the activation action is a thermal stress combined with a compression stress or shear stress, the thermal stress configured to reduce the predetermined activation threshold of the compression stress or the shear stress.

12. The activatable temperature exposure indicator of claim 1, wherein the bonding material comprises a material selected from a group consisting of: a polymer having side-chain crystallinity, polymeric materials, an alkane, a wax, an alkane wax, and combinations thereof.

13. The activatable temperature exposure indicator of claim 1, wherein the indicator material is selected from a group consisting of a polymer having side-chain crystallinity, polymeric materials, an alkane, a wax, an alkane wax, dyes, leuco dyes, chemical pigments, particles containing copper, particles containing silver, particles containing graphite, particles containing conductive metals, particles containing conductive non-metal materials, and combinations thereof.

14. The activatable temperature exposure indicator of claim 1, wherein the activatable shells comprise a material selected from a group consisting of: a protein, a gel, a polyurea formaldehyde, a polymelamine formaldehyde, a wax material, an emulsion, and combinations thereof.

15. The activatable temperature exposure indicator of claim 1, wherein the response temperature is defined within a range bounded by −15 degrees Celsius (C) and 100 35 degrees C.

16. The activatable temperature exposure indicator of claim 1, wherein the liquefaction temperature of the bonding material is greater than the response temperature of the activatable temperature exposure indicator.

17. The activatable temperature exposure indicator of claim 1, wherein the liquefaction temperature of the bonding material is defined within a range bounded by 40 degrees Celsius (C) and 110 115 degrees C.

18. (canceled)

19. A method of making an activatable temperature exposure indicator, comprising:
placing a wick on a substrate; and
dispensing a bonding material proximately to a wick;
wherein a plurality of microcapsules is dispersed within the bonding material, each microcapsule including an indicator material contained within an activatable shell,
wherein the bonding material containing the microcapsules is dispensed at a dispensation temperature above a liquefaction temperature of the bonding material, such that the bonding material is dispensed in a liquid phase,
wherein the bonding material containing the microcapsules transitions out of the liquid phase after being dispensed and contacting the wick,
wherein the bonding material couples the microcapsules to the wick without filling pores of the wick, and
wherein the bonding material blocks migration of the microcapsules into the wick.

20. The method of claim 19, wherein the liquefaction temperature of the bonding material is greater than a response temperature of the indicator material and defined within a range bounded by 40 degrees C. and 120 degrees C.

21. (canceled)

22. The method of claim 19, wherein the bonding material comprises a material selected from a group consisting of: a polymer having side-chain crystallinity, polymeric materials, an alkane, a wax, an alkane wax, and combinations thereof.

23. The method of claim 19, wherein the indicator material is configured to liquefy responsive to an exposure to a temperature above a predetermined response temperature defined within a range bounded by −15 degrees Celsius (C) and 100 degrees C.

24. The method of claim 23, wherein the activatable shells are configured to contain the indicator material when liquefied.

25. The method of claim 24, wherein the activatable shells are configured to rupture in response to an application of an activation action exceeding a predetermined activation threshold, releasing the indicator material.

26. The method of claim 25, wherein the indicator material is configured to permeate pores of the wick and migrate along the wick into an indicator region when liquefied and released from the microcapsules, producing an observable change in the indicator region.

27-28. (canceled)

29. The method of claim 19, wherein the indicator material is selected from a group consisting of: a polymer having side-chain crystallinity, polymeric materials, an alkane, a wax, an alkane wax, dyes, leuco dyes, chemical pigments, particles containing copper, particles containing silver, particles containing graphite, particles containing conductive metals, particles containing conductive non-metal materials, and combinations thereof.

30-31. (canceled)

32. The method of claim 19, wherein the wick comprises a laminate layer and a wicking material, and the method further comprising:
cutting a laminated sheet of the laminate layer and the wicking material into a plurality of wicks;
picking the wick from the plurality of wicks using a pick and place device; and
placing the wick of the plurality of wicks onto the substrate using the pick and place device.

33-38. (canceled)

39. An activatable environmental exposure indicator having a response condition, comprising:
a substrate;
a wick physically coupled to or contained in the substrate;
an indicator region, on or adjacent to a first end of the wick; and
a bonding material containing a plurality of microcapsules disposed on or adjacent to a second end of the wick, opposite the first end;
wherein each microcapsule of the plurality of microcapsules contains an indicator material microencapsulated in an activatable shell,
wherein the indicator material is configured to liquefy responsive to exposure to the response condition,
wherein the activatable shells are configured to contain the indicator material when the liquefied,
wherein the activatable shells are configured to rupture in response to an application of an activation action exceeding a predetermined activation threshold, releasing the indicator material,
wherein the indicator material is configured to permeate pores of the wick and migrate along the wick into the indicator region when liquefied and released from the microcapsules, producing an observable change in the indicator region,
wherein the bonding material couples the microcapsules to the wick without filling pores of the wick, and
wherein the bonding material facilitates migration of liquefied indicator material into the wick, and blocks migration of the microcapsules into the wick.

40. The activatable environmental exposure indicator of claim 39, wherein the response condition is selected from a group consisting of: a temperature excursion above a predetermined temperature, a temperature excursion above a predetermined temperature threshold for at least a predetermined amount of time, a temperature excursion below a predetermined temperature, a temperature excursion below a predetermined temperature for at least a predetermined amount of time, cumulative exposure to temperature over a time period above a predetermined threshold for at least a predetermined amount of time, an exposure to a particular chemical, an oxygen exposure, an ammonia exposure, an exposure to a particular chemical above a threshold concentration, an exposure to a particular chemical above the threshold concentration for at least a predetermined amount of time, an exposure to at least a predetermined amount of radiation of a particular type, an predetermined electromagnetic exposure, a humidity exposure, an exposure to a humidity level above a predetermined threshold, and an exposure to a humidity level above a predetermined threshold for at least a predetermined amount of time.
